(19) United States
(12) Patent Application Publication
(10) Pub. No.: US 2026/0000346 A1
(43) Pub. Date: Jan. 1, 2026

(54) FLEXION/EXTENSION SURGICAL GUIDES AND METHODS OF USING THE SAME

(71) Applicant: WRIGHT MEDICAL TECHNOLOGY, INC., Memphis, TN (US)

(72) Inventors: Meghan R. KUBACKI, Cookeville, TN (US); Kian-Ming WONG, Lakeland, TN (US)

(73) Assignee: WRIGHT MEDICAL TECHNOLOGY, INC., Memphis, TN (US)

(21) Appl. No.: 19/329,612

(22) Filed: Sep. 16, 2025

Related U.S. Application Data
(62) Division of application No. 18/179,488, filed on Mar. 7, 2023, now Pat. No. 12,433,532.
(60) Provisional application No. 63/365,717, filed on Jun. 2, 2022.

(51) Int. Cl. A61B 5/00; A61B 5/107

(52) U.S. Cl. CPC A61B 5/4595; A61B 5/1071; A61B 5/6829

(57) ABSTRACT

A surgical guide includes a body having four sides. The body comprises a first base at the first side, a second base at the second side and an elongated extension between the first and second base. The first base defines at least one first hole and the second base defines at least a second hole. The body is sized and configured to be installed above a joint line of a calcaneus and a talus bone and approximately mid-way along a longitudinal axis of a tibia. The first base is configured to receive at least one dowel of a second surgical guide in the at least one first hole and the second base is configured to receive at least one pin sleeve and pin in the second hole. Systems and methods are also disclosed.

### CROSS-REFERENCE TO RELATED APPLICATION

This application claims priority under 35 U.S.C. § 119 (e) to U.S. Provisional Application No. 63/365,717, filed on Jun. 2, 2022, the entire contents of which are incorporated herein by reference.

### INCORPORATION BY REFERENCE

The disclosures of U.S. Pat. Nos. 9,918,724 and 10,136,904 are incorporated by reference herein in their entireties.

### FIELD OF DISCLOSURE

The disclosed apparatuses, systems, and methods relate to surgical tools. More specifically, the disclosed apparatuses, systems, and methods relate to surgical tools for determining the flexion and/or extension angle when performing ankle surgery, including total ankle surgery.

### BACKGROUND

The ankle is a joint that is often compared to a hinge. This joint is made from the combination of three bones—the tibia, fibula and talus. The talus is the ankle bone. The upper portion of the talus fits inside a socket that is formed by the lower portion of the leg, which includes the tibia and the fibula. Ankle joint deterioration can develop from arthritis, bone degeneration, and/or injury, which may result in pain, lower range of motion, and decreased quality of life. To treat these conditions, physicians often recommend ankle replacement surgery with an implant. One example of such an implant is the INFINITY™ Total Ankle System available from Stryker Corp., of Kalamazoo, MI, although one of ordinary skill in the art will understand that the disclosure is not limited to such implants. The process of implanting an ankle replacement system typically includes the use of guides for determining the proper placement and angle of the implant, among other surgical guides and tools.

### SUMMARY

The disclosed guides, systems, and methods reduce the number of surgical components and simplify the manner in which a surgeon determines the flexion and/or extension angle of an ankle implant, as compared to conventional instrumentation. The disclosed guides, systems, and methods result in a more streamlined and efficient method for determining the proper placement and angle of an ankle implant. Additionally, the disclosed guides, systems, and methods are smaller in size, which allows for improved manipulation of the guides during surgery. Further, the disclosed guides, systems, and methods provide enhanced visibility cues which allow for greater accuracy in placement of the guides and ankle implant during surgery.

In some embodiments, the disclosed surgical guide includes a first side, a second side, a third side, and a fourth side, with the third and fourth sides extending between the first and second sides. The body comprises a first base at the first side, a second base at the second side and an elongated extension between the first and second base. The first base defines at least one first hole and the second base defines at least a second hole. The body is sized and configured to be installed above a joint line of a calcaneus and a talus bone and approximately mid-way along a longitudinal axis of a tibia. The first base is configured to receive at least one dowel of a second surgical guide in the at least one first hole and the second base is configured to receive at least one pin sleeve and pin in the second hole.

In some embodiments, the first base includes a posterior side comprising a protrusion configured to engage a channel of a third surgical guide.

In some embodiments, the first base includes a posterior side comprising a channel configured to engage a side of a third surgical guide.

In some embodiments, the first base includes an anterior side comprising an adjustment mechanism to lock the third surgical guide and the surgical guide.

In some embodiments, the first and second base are rectangular in shape.

In some embodiments, the first base is configured to receive at least one pin in the at least one first hole.

In some embodiments, the second base includes a superior side and an inferior side, as well as an engagement portion disposed within a channel configured to allow movement of the engagement portion between the superior side and inferior side. The engagement portion defines at least the second hole configured to receive the at least one pin sleeve and pin.

In some embodiments, the second base includes an adjustment mechanism to lock the engagement portion in place.

In some embodiments, the engagement portion is rectangular in shape.

In some embodiments, the engagement portion includes an adjustment mechanism configured to lock a pin in place.

In some embodiments, the pin sleeve includes at least one slot configured to engage a portion of the adjustment mechanism configured to lock a pin in place.

In some embodiments, the pin sleeve comprises two slots, in which one slot is located on the superior side of the pin sleeve and one slot is located on the inferior side of the pin sleeve. Each slot is configured to engage a portion of the adjustment mechanism configured to lock a pin in place in either configuration of the pin sleeve.

In some embodiments, the pin sleeve is rectangular in shape and the second hole is similarly configured to receive the rectangular shape of the pin sleeve.

In some embodiments, at least a portion of the body is formed from radiopaque material.

In some embodiments, the disclosed system comprises a first guide including a body having a first side, a second side, a third side, and a fourth side, with the third and fourth sides extending between the first and second sides. The body includes a first base at the first side, a second base at the second side and an elongated extension between the first and second base. The first base defines at least one first hole and the second base defines at least a second hole. The body is sized and configured to be installed above a joint line of a calcaneus and a talus bone and approximately mid-way along the longitudinal axis of a tibia. The first base is configured to receive at least one protrusion of a second surgical guide in the at least one first hole and the second base is configured to receive at least one pin sleeve and pin in the second hole.

In some embodiments, the disclosed system includes a second guide having a transverse beam that extends between a first arm and a second arm. The second guide is configured to be coupled to the first guide.

In some embodiments, first and second protrusions extend from the transverse beam of the second guide. The first and second protrusions are sized and configured to be received in the at least one first hole defined by the first base for coupling the second guide to the first guide.

In some embodiments, each of the first and second arms of the second guide define at least one of a hole or a slot for receiving an elongate radiopaque device therein.

In some embodiments, the disclosed system includes a third guide having a first portion extending longitudinally in a first direction and a second portion extending laterally from the first portion in a second direction. The first portion defines a first channel that runs the length of first portion on a first side and defines a second channel on a second side, which is disposed opposite the first side.

In some embodiments, the second portion of the third guide defines a guide hole which is configured to engage a shaft of a fourth surgical guide inserted above a joint line of a calcaneus and a talus bone.

In some embodiments, the first base of the first guide includes a posterior side comprising a protrusion configured to engage a channel of the third guide.

In some embodiments, the first base of the first guide includes a posterior side comprising a channel configured to engage a side of the third surgical guide.

In some embodiments, the first base of the first guide includes an anterior side comprising an adjustment mechanism to align the third guide and the first guide.

In some embodiments, the disclosed method includes: inserting a first guide above a joint line of a calcaneus and a talus bone; connecting a second guide to the first guide, the second guide having a first portion extending longitudinally in a first direction and a second portion extending laterally from the first portion in a second direction, wherein the first portion defines a first channel that runs the length of first portion on a first side and defines a second channel on a second side, which is disposed opposite the first side, and the second portion of the second guide defines a guide hole which is configured to engage a shaft of the first guide; connecting a portion of the second guide to a third guide, the third guide including a body having a first side, a second side, a third side, and a fourth side, the third and fourth sides extending between the first and second sides, wherein the body comprises a first base at said first side, a second base at said second side and an elongated extension between the first and second base, wherein the first base defines at least one first hole and the second base defines at least a second hole, and wherein the body is sized and configured to be installed above a joint line of a calcaneus and a talus bone and approximately mid-way along the longitudinal axis of a tibia; locking the location of the second guide and third guide about an axis defined by a shaft of the tibia using an adjustment mechanism attached to the third guide; coupling a fourth guide to the third guide, the fourth guide including first and second arms that are coupled together by a transverse beam and each of the first and second arms of the fourth guide define at least one of a hole or a slot for receiving an elongate radiopaque device therein; coupling an elongate radiopaque device to the fourth guide through the at least one hole or slot of the fourth guide; determining the alignment of the fourth guide and elongate radiopaque device using fluoroscopy; inserting at least one pin sleeve and pin into the second hole of the third guide; removing the fourth guide and elongate radiopaque device; inserting pins into the at least one first hole of the third guide and through two cortices of the tibia; and removing the first, second and third guides.

In some embodiments, coupling the fourth guide to the third guide includes inserting at least one protrusion extending from the transverse beam of the fourth guide into at least one hole defined by the third guide.

In some embodiments, inserting at least one pin into the second hole of the third guide further includes turning an adjustment mechanism located on the second base to lock the pin in place after inserting the pin sleeve and pin into the second hole of the third guide.

In some embodiments, the disclosed method includes loosening all adjustment mechanisms attached to the third guide prior to removing the first, second and third guides.

In some embodiments, the disclosed method includes: inserting a first guide above a joint line of a calcaneus and a talus bone; connecting a portion of a second guide to a third guide, the second guide having a first portion extending longitudinally in a first direction and a second portion extending laterally from the first portion in a second direction, wherein the first portion defines a first channel that runs the length of first portion on a first side and defines a second channel on a second side, which is disposed opposite the first side, and the second portion of the second guide defines a guide hole which is configured to engage a shaft of the first guide, wherein the third guide includes a body having a first side, a second side, a third side, and a fourth side, the third and fourth sides extending between the first and second sides, wherein the body comprises a first base at said first side, a second base at said second side and an elongated extension between the first and second base, wherein the first base defines at least one first hole and the second base defines at least a second hole, and wherein the body is sized and configured to be installed above a joint line of a calcaneus and a talus bone and approximately mid-way along the longitudinal axis of a tibia; inserting at least one pin sleeve into the second hole of the third guide; coupling a fourth guide to the third guide, the fourth guide including first and second arms that are coupled together by a transverse beam and each of the first and second arms of the fourth guide define at least one of a hole or a slot for receiving an elongate radiopaque device therein; coupling an elongate radiopaque device to the fourth guide through the at least one hole or slot of the fourth guide; connecting the second guide to the first guide; locking the location of the second guide and third guide about an axis defined by a shaft of the tibia using an adjustment mechanism attached to the third guide; determining the alignment of the fourth guide and elongate radiopaque device using fluoroscopy; inserting at least one pin into the pin sleeve that is inserted into the second hole of the third guide; removing the fourth guide and elongate radiopaque device; inserting pins into the at least one first hole of the third guide and through two cortices of the tibia; and removing the first, second and third guides.

### BRIEF DESCRIPTION OF THE DRAWINGS

The features and advantages of the present disclosures will be more fully disclosed in, or rendered obvious by the following detailed descriptions of example embodiments. The detailed descriptions of the example embodiments are to be considered together with the accompanying drawings wherein like numbers refer to like parts and further wherein:

FIG. 1 is an isometric perspective view of one example of a guide in accordance with some embodiments;

FIG. 2 is a front or anterior side view of the guide illustrated in FIG. 1 in accordance with some embodiments;

FIG. 3 is a rear or posterior side view of the guide illustrated in FIG. 1 in accordance with some embodiments;

FIG. 4A is a side view of the guide illustrated in FIG. 1 in accordance with some embodiments;

FIG. 4B is a cross-sectional view of guide 100 taken along its vertical axis, in accordance with some embodiments;

FIG. 5 is a side view of the guide illustrated in FIG. 1 that is opposite the side view of FIG. 4 in accordance with some embodiments;

FIG. 6 is a top or superior side view of the guide illustrated in FIG. 1 in accordance with some embodiments;

FIG. 7A is a bottom or inferior side view of the guide illustrated in FIG. 1 in accordance with some embodiments;

FIG. 7B is a cross-sectional view of the guide illustrated in FIG. 1, taken long its longitudinal axis, in accordance with some embodiments;

FIG. 8 is a top side view of one example of a medial gutter fork in accordance with some embodiments;

FIG. 9 is a rear end view of the medial gutter fork illustrated in FIG. 8 in accordance with some embodiments;

FIG. 10 is a front end view of the medial gutter fork illustrated in FIG. 8 in accordance with some embodiments;

FIG. 11 is a top side view of a rotation guide slide in accordance with some embodiments;

FIG. 12 is a rear end view of the rotation guide slide illustrated in FIG. 8 in accordance with some embodiments;

FIG. 13 is a side profile view of the rotation guide slide illustrated in FIG. 8 in accordance with some embodiments;

FIG. 14 is an isometric view of the rotation guide slide illustrated in FIG. 11 coupled to the guide illustrated in FIG. 1 and the medial gutter fork illustrated in FIG. 8, in accordance with some embodiments;

FIG. 15 is a side view of the rotation guide slide illustrated in FIG. 11 coupled to the guide illustrated in FIG. 1 and the medial gutter fork illustrated in FIG. 8, in accordance with some embodiments;

FIG. 16 is a top side view of one example of an alignment guide in accordance with some embodiments;

FIG. 17 is a rear end view of the alignment guide illustrated in FIG. 16 in accordance with some embodiments;

FIG. 18 is a front end view of the alignment guide illustrated in FIG. 16 in accordance with some embodiments;

FIG. 19 is an isometric view of the alignment guide illustrated in FIG. 16 coupled to the guide illustrated in FIG. 1, in accordance with some embodiments;

FIG. 20 is a side view of the alignment guide illustrated in FIG. 16 coupled to the guide illustrated in FIG. 1, in accordance with some embodiments;

FIG. 21 is a top side view of another example of an alignment guide in accordance with some embodiments;

FIG. 22 is a distal end view of the alignment guide illustrated in FIG. 21 in accordance with some embodiments;

FIG. 23 is a proximal end view of the alignment guide illustrated in FIG. 21 in accordance with some embodiments;

FIG. 24 is an isometric view of the alignment guide illustrated in FIG. 21 coupled to the alignment guide illustrated in FIG. 16, which is coupled to the guide illustrated in FIG. 1, which is coupled to the rotation guide slide illustrated in FIG. 11, which is coupled to the medial gutter fork illustrated in FIG. 8, in accordance with some embodiments;

FIG. 25 is a side view of the alignment guide illustrated in FIG. 21 coupled to the alignment guide illustrated in FIG. 16, which is coupled to the guide illustrated in FIG. 1, which is coupled to the rotation guide slide illustrated in FIG. 11, which is coupled to the medial gutter fork illustrated in FIG. 8, in accordance with some embodiments;

FIG. 26 is an isometric view of one example of a pin sleeve that may be used with the guide illustrated in FIG. 1 in accordance with some embodiments;

FIG. 27 is a front end view of the pin sleeve illustrated in FIG. 26 in accordance with some embodiments;

FIG. 28 is a rear end view of the pin sleeve illustrated in FIG. 26 in accordance with some embodiments;

FIG. 29 is an isometric view of another example of a pin sleeve that may be used with the guide illustrated in FIG. 1 in accordance with some embodiments;

FIG. 30 is an isometric view of one example of a pin that may be used with the guide illustrated in FIG. 1 in accordance with some embodiments;

FIG. 31 is an isometric view of one example of a trocar that may be used with the guide illustrated in FIG. 1 in accordance with some embodiments;

FIG. 32 is an isometric view of the pin sleeve illustrated in FIG. 29 and a pin illustrated in FIG. 30 disposed within a hole defined by the proximal end of the guide illustrated in FIG. 1, which is coupled to the alignment guides as illustrated in FIGS. 16 and 21, in accordance with some embodiments;

FIG. 33 is a side view of the pin sleeve illustrated in FIG. 29 and a pin illustrated in FIG. 30 disposed within the hole defined by the proximal end of the guide illustrated in FIG. 1, which is coupled to the alignment guides as illustrated in FIGS. 16 and 21, in accordance with some embodiments;

FIG. 34 is an isometric view of a plurality of pins disposed within pin sleeves illustrated in FIG. 26, which are disposed within a plurality of holes defined by the distal end of the guide illustrated in FIG. 1, and a pin illustrated in FIG. 30 disposed within the pin sleeve illustrated in FIG. 29, which is disposed within the hole defined by the proximal end of the guide illustrated in FIG. 1, in accordance with some embodiments;

FIG. 35 is a side view of a plurality of pins disposed within pin sleeves illustrated in FIG. 26, which are disposed within a plurality of holes defined by the distal end of the guide illustrated in FIG. 1, and a pin illustrated in FIG. 30 disposed within the pin sleeve illustrated in FIG. 29, which is disposed within the hole defined by the proximal end of the guide illustrated in FIG. 1, in accordance with some embodiments.

### DETAILED DESCRIPTION

This description of the exemplary embodiments is to be read in connection with the accompanying drawings, which are to be considered part of the entire written description. The drawing figures are not necessarily to scale and certain features of the invention may be shown exaggerated in scale or in somewhat schematic form in the interest of clarity and conciseness. In the description, relative terms such as “horizontal,” “vertical,” “up,” “down,” “top,” and “bottom” as well as derivatives thereof (e.g., “horizontally,” “downwardly,” “upwardly,” etc.) should be construed to refer to the orientation as then described or as shown in the drawing figure under discussion. These relative terms are for convenience of description and normally are not intended to require a particular orientation. Terms including “inwardly” versus “outwardly,” “longitudinal” versus “lateral” and the like are to be interpreted relative to one another or relative to an axis of elongation, or an axis or center of rotation, as appropriate. Terms concerning attachments, coupling and the like, such as “connected” and “interconnected,” refer to a relationship wherein structures are secured or attached to one another either directly or indirectly through intervening structures, as well as both movable or rigid attachments or relationships, unless expressly described otherwise.

The disclosed guides reduce the number of components and provides a simpler manner of determining the flexion and/or extension angle of an ankle implant compared to conventional instrumentation, which yields a more streamlined and efficient method. The disclosed guides also have a reduced size, which improves the ability of the surgeon to manipulate the guides during surgery. Further, the disclosed guides include enhanced visibility cues for accurate placement during the surgery.

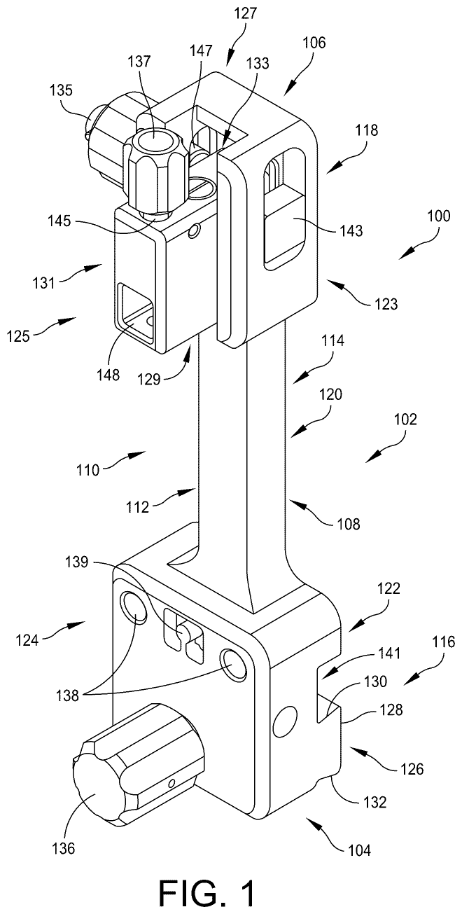

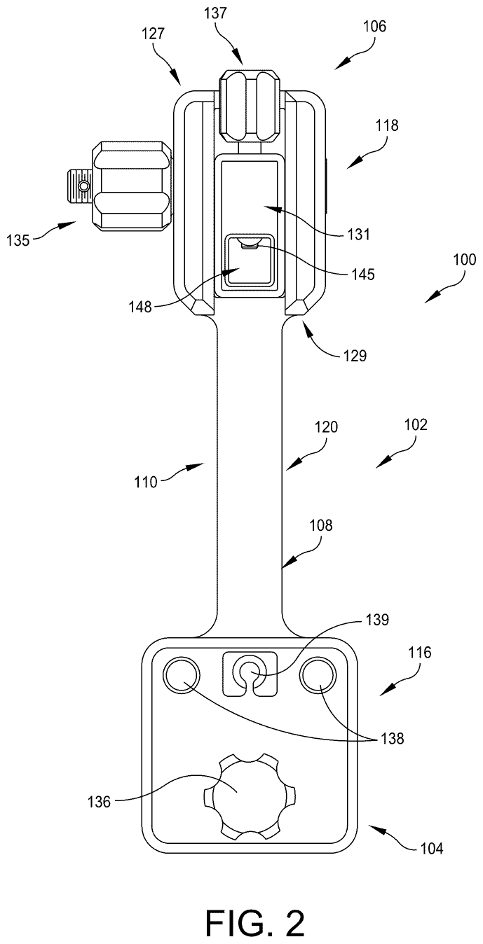

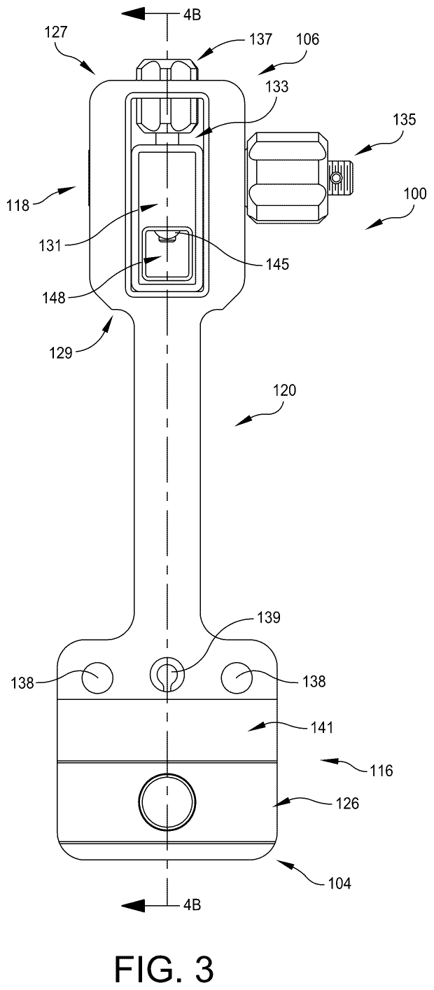

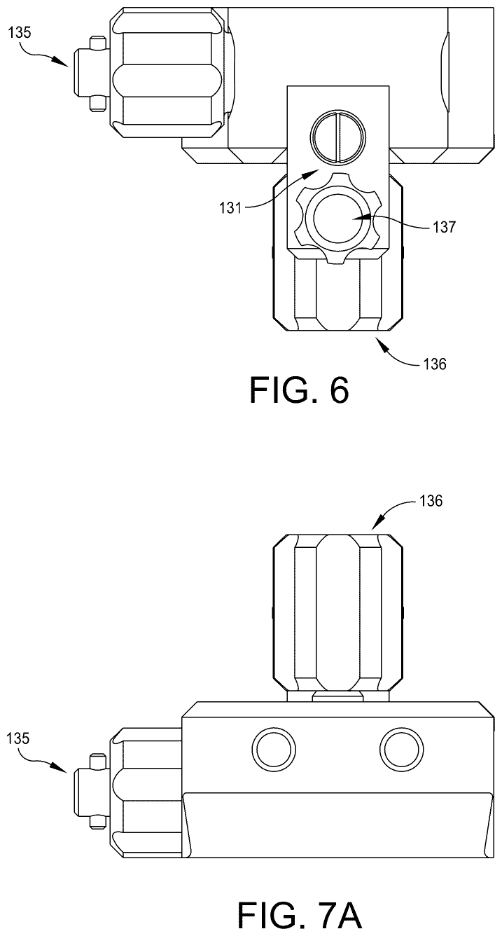

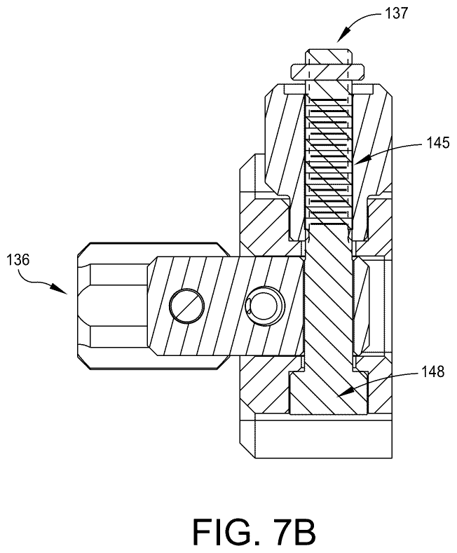

FIGS. 1-7 illustrate one example of a flexion/extension guide 100 in accordance with some embodiments. Guide 100 includes a body 102 extending from an inferior side 104 to a superior side 106, as seen in FIGS. 6 and 7. Body 102 further includes opposite sides 108, 110, an anterior side 112, and a posterior side 114, as seen in FIGS. 2 and 3.

In some embodiments, body 102 may be formed from a rigid radiopaque material, such as a surgical grade metal.

Body 102 comprises a wide, rectangular base 116 on the inferior side 104, a narrower, rectangular base 118 on the superior side 106, and a narrow, elongated extension 120 between bases 116 and 118. In some embodiments, body 102 is sized and configured to be installed above a joint line of a calcaneus and a talus bone, such as between a medial gutter of an ankle joint and approximately mid-way along the longitudinal axis of a tibia 260. One of ordinary skill in the art will understand that while the bases 116 and 118 are shown as having a generally rectangular shape, the bases 116 and 118 may have other shapes, e.g., square, etc.

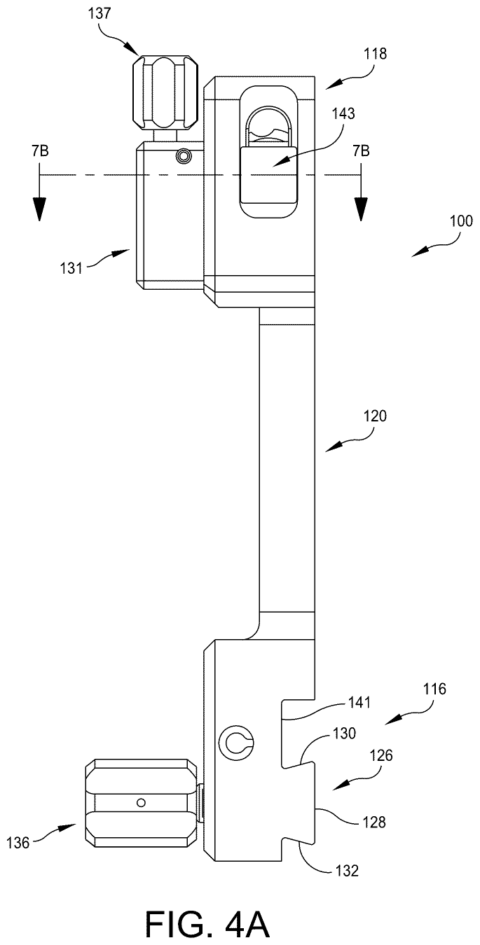

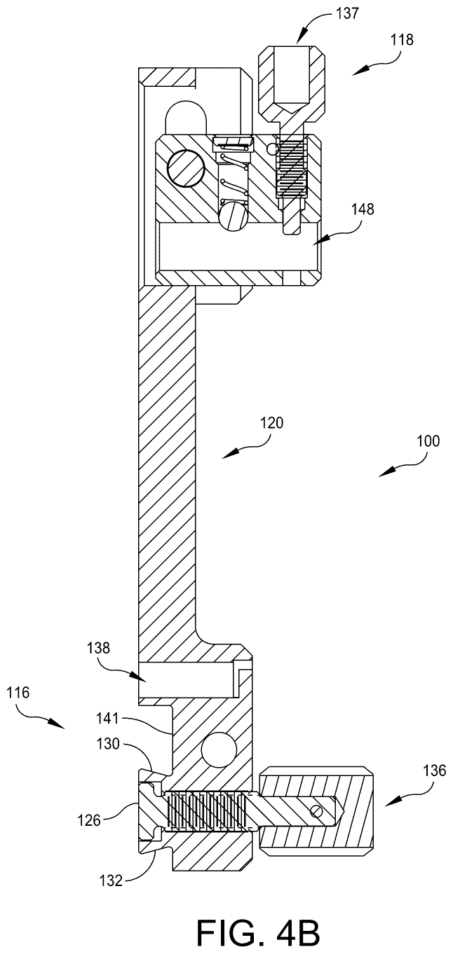

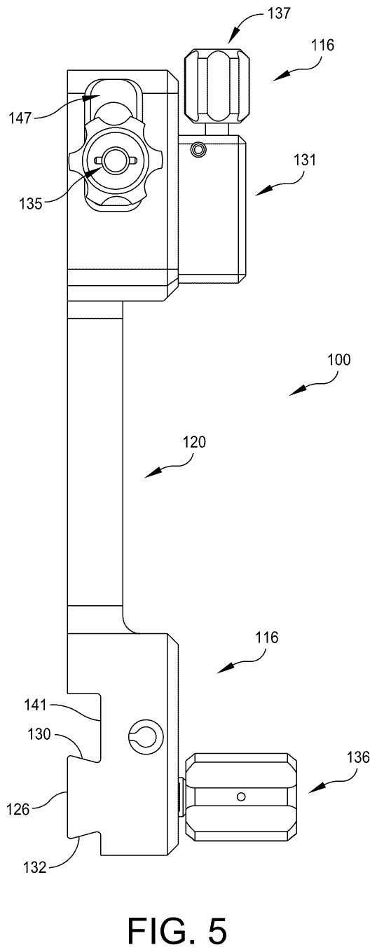

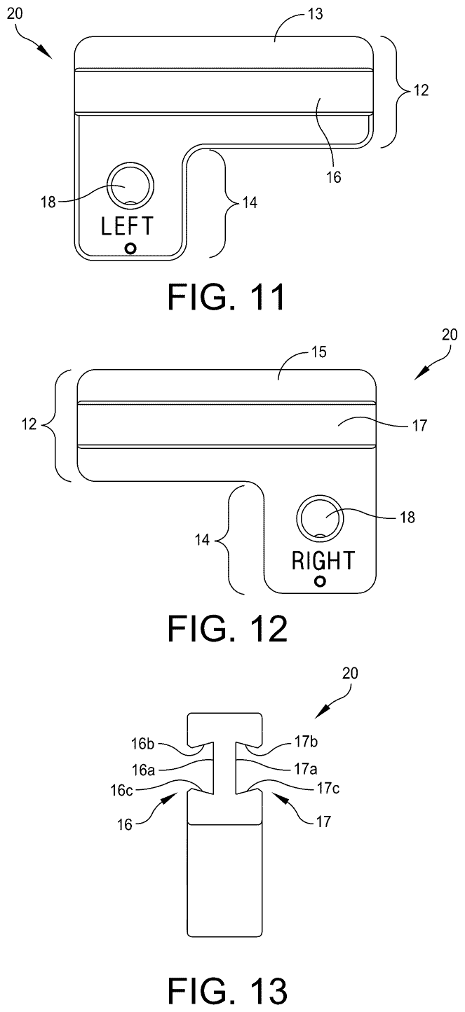

Base 116 includes a posterior side 122 and anterior side 124. Posterior side 122 of base 116 may include a protrusion 126 configured to engage a first channel 16 or second channel 17 of a rotation guide slide 20, as shown in FIGS. 11-13. Protrusion 126 extends longitudinally across base 116 (i.e., in a direction that is perpendicular with respect to the longitudinal length of elongated extension 120). As best seen in FIGS. 4 and 5, protrusion 126 has a flat bottom surface 128 and angled sides 130 and 132 such that the top portion of protrusion 126 is narrower than the bottom surface such that protrusion 126 has a complementary shape to channels 16 and 17 of rotation guide slide 20. In some embodiments, sides 130 and 132 of protrusion 126 perpendicularly extend from bottom surface 128 of protrusion 126. One of ordinary skill in the art will understand that while protrusion 126 is shown as having a generally flat bottom shape and continuous, protrusion 126 may have other shapes, e.g. T-slot shaped, shaped with an interruption such as a groove dividing protrusion 126 into two portions, etc.

In some embodiments, the posterior side 122 of base 116 may also include a channel 141 configured to engage side 22 or 24 of rotation guide slide 20, as described in greater detail below.

The anterior side 124 of base 116 may include an adjustment mechanism, such as knob 136 to adjust the combined rotation guide slide 20 and flexion/extension guide 100 so that extension 120 is approximately aligned with the mechanical axis of the tibia 260. Knob 136 may be connected to a screw, which locks protrusion 126 into either first channel 16 or second channel 17.

Base 116 may also include a at least one hole 138, configured to receive dowels or protrusions 210-1, 210-2 (collectively, “dowels 210” or “protrusions 210”) of the angel wing alignment guide 200 as well as pins 140, 142 disposed in pin sleeves 144, 146 in accordance with some embodiments. Base 116 may also include hole 139 between at least one hole 138. Hole 139 may be used as an alignment visual cue, which provides information regarding whether the view is true and not skewed using an x-ray. One of ordinary skill in the art will understand that while at least one hole 138 is shown as having a generally round shape, at least one hole 138 may have other shapes, e.g., oblong or slot-shaped.

Base 118 includes a posterior side 123 and anterior side 125, as well as a superior side 127 and inferior side 129. In some embodiments, base 118 may include a channel 133, configured to allow movement of a rectangular engagement portion 131 disposed therein between superior side 127 and inferior side 129. Rectangular engagement portion 131 may have the ability to slide and rotate within channel 133 to enable guide 100 to set the flexion and extension angle of an ankle implant. Base 118 may also include hole or channel 147 on side 108 and a knob 135 disposed therein. Knob 135 may be configured to engage a block portion 143 on side 110 of base 118 to lock rectangular engagement portion 131 in place.

Rectangular engagement portion 131 of base 118 may include a hole 148 from the anterior side 125 to the posterior side 123, that is configured to receive pin 150 disposed in a pin sleeve 152. In some embodiments, pin sleeve 152 is rectangular and hole 148 is similarly configured to receive the rectangular shape of pin sleeve 152. Rectangular engagement portion 131 of base 118 may also include a knob 137 and extension 145 on the superior side 127 that may be rotated to allow extension 145 to be inserted into slot 154 of rectangular pin sleeve 152 to lock pin 150 in place. In some embodiments, rectangular pin sleeve 152 may have two slots 154, wherein one slot located on the superior side of the pin sleeve and one slot located on the inferior side of the pin sleeve, thereby allowing rectangular pin sleeve 152 to be inserted in either configuration and providing alignment with extension 145 in either configuration.

Knobs 135, 136 and 137 may be connected to screws that engage and lock their respective portions of guide 100, as shown in FIGS. 4B and 7B, which illustrate sections B-B and C-C of guide 100, taken along its longitudinal and vertical axes. Additionally, as shown in FIG. 4B, a ball and spring plunger may be used to retain rectangular pin sleeve 152.

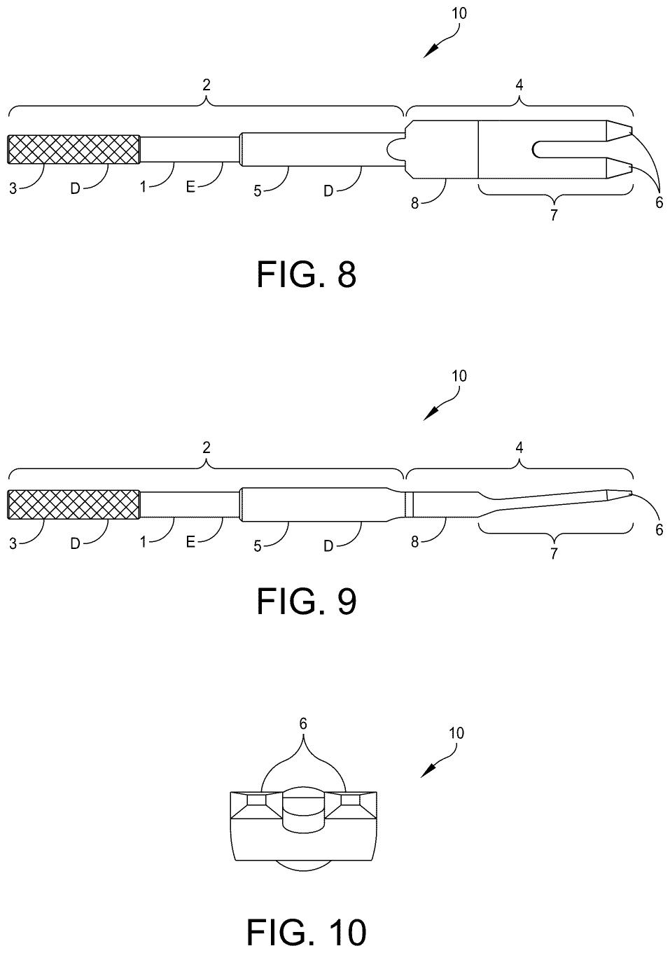

Referring to FIGS. 8-10, a medial gutter fork 10 includes a shaft 2 and a head 4. In some embodiments, shaft 2 has a cylindrical geometry and includes a proximal end 3 and a distal end 5 each being of a first diameter D, and an inner section 1 disposed between proximal end 3 and distal end 5 and having a second diameter E. In some embodiments, the first diameter D is greater than second diameter E. Head 4 has a transitional portion 8, which is connected to the distal end 5 of shaft 2, and a forked portion 7 including a pair of prongs 6. Medial gutter fork 10 is configured to be inserted into the medial gutter of an ankle joint to serve as a reference point to additional elements of guides for determining the proper placement and angle of an ankle implant. In some embodiments, the head 4 does not have a forked shape. One of ordinary skill in the art will understand that the angle of an ankle implant may include 0 degrees.

Referring to FIGS. 11-13, rotation guide slide 20 has an “L” shaped body including a first portion 12 extending longitudinally in a first direction and a second portion 14 extending laterally from the first portion 12 in a second direction. In some embodiments, first portion 12 is longer than second portion 14 and the first and second directions are perpendicular with respect to one another. One of ordinary skill in the art will understand that while first portion 12 is shown as longer than second portion 14, first portion 12 may be a different length, e.g., equal to or shorter than second portion 14. First portion 12 defines a first channel 16 that runs the length of first portion 12 on a first side 13 and defines a second channel 17 on second side 15, which is disposed opposite the first side 13. Second portion 14 defines a guide hole 18 which is configured to engage the shaft 2 of medial gutter fork 10. In some embodiments, hole 18 is located at the approximate center of second portion 14; however, hole 18 can be located at other positions of second portion 14.

FIG. 13 illustrates one example of a configuration of channel 16 in accordance with some embodiments. Channel 16 is illustrated as having a flat bottom surface 16 a and angled side walls 16 b and 16 c that taper inwardly such that the top of channel 16 is narrower than the bottom. Second channel 17 is aligned along the same longitudinal axis as first channel 16 and is shaped identical to first channel 16 with a flat bottom surface 17 a and angled side walls 17 b and 17 c that taper inwardly such that the top of channel 17 is narrower than the bottom. One of ordinary skill in the art will understand that while channels 16 and 17 are shown as having generally flat bottom shapes, channels 16 and 17 may have other shapes, e.g. T-slot shaped, etc.

Either first channel 16 or second channel 17 faces away from the ankle and will engage with flexion/extension guide 100 as described in greater detail below. This configuration enables rotation guide slide 20 to be used during an ankle replacement procedure for either the left ankle or right ankle.

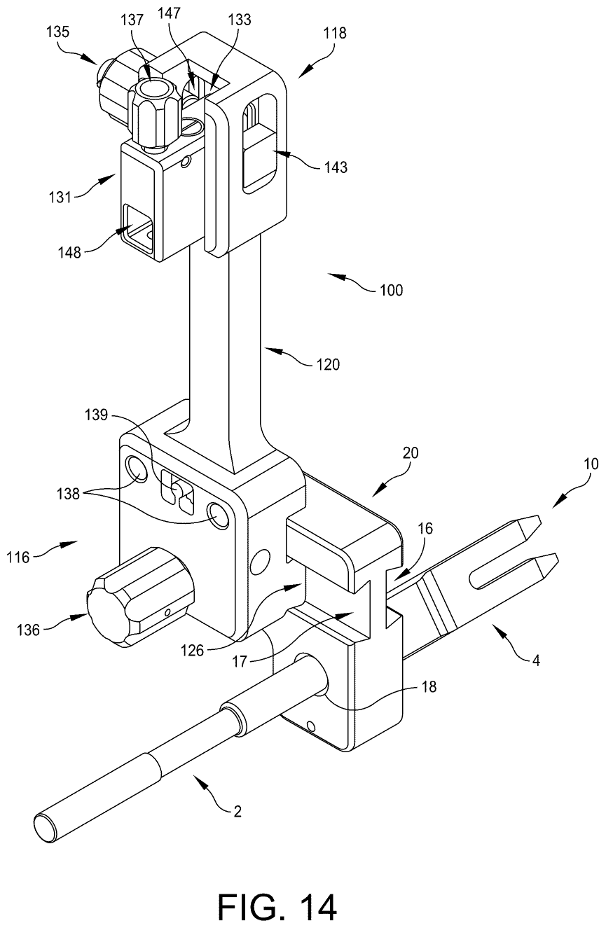

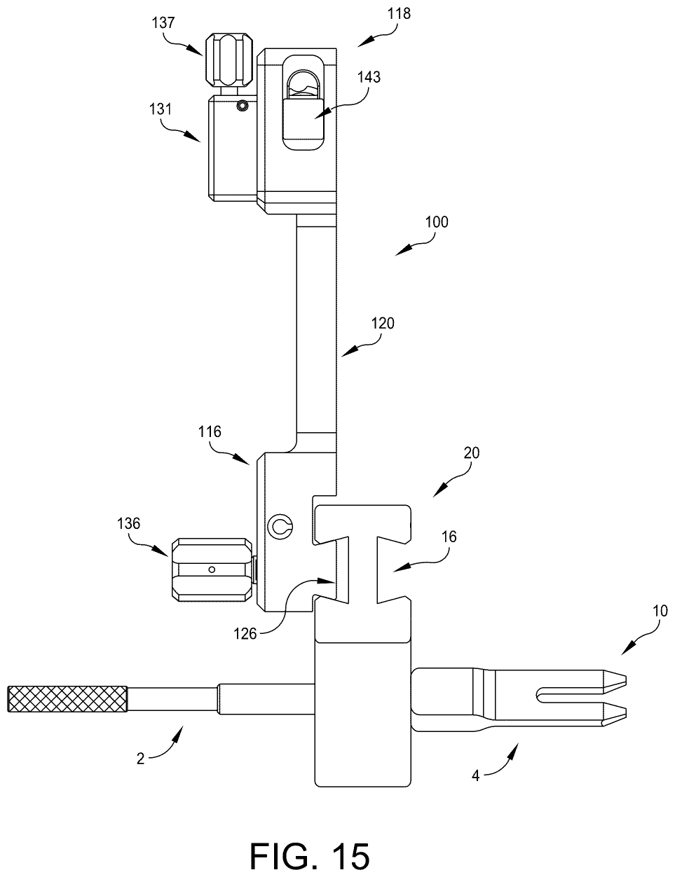

The guide 100 with medial gutter fork 10 and rotation guide slide 20 is now briefly described with reference to FIGS. 14-15, which is used once access is gained to the tibia 260 and talus 265.

Gutter fork 10 is inserted into the medial gutter of the ankle joint, and rotation guide slide 20 is operationally connected to medial gutter fork 10 by placing guide hole 18 over distal end 5 of shaft 2 as illustrated in FIG. 14. Rotation guide slide 20 is positioned with either first channel 16 or second channel 17 facing away from the tibia 260. Flexion/extension guide 100 is operationally connected to rotation guide slide 20 by sliding protrusion 126 into either first channel 16 or second channel 17, whichever is facing away from the tibia 260. Thus assembled, an operator uses knob 136 to lock the mediolateral translation location of rotation guide slide 20 and flexion/extension guide 100 about an axis defined by the shaft of the tibia 260.

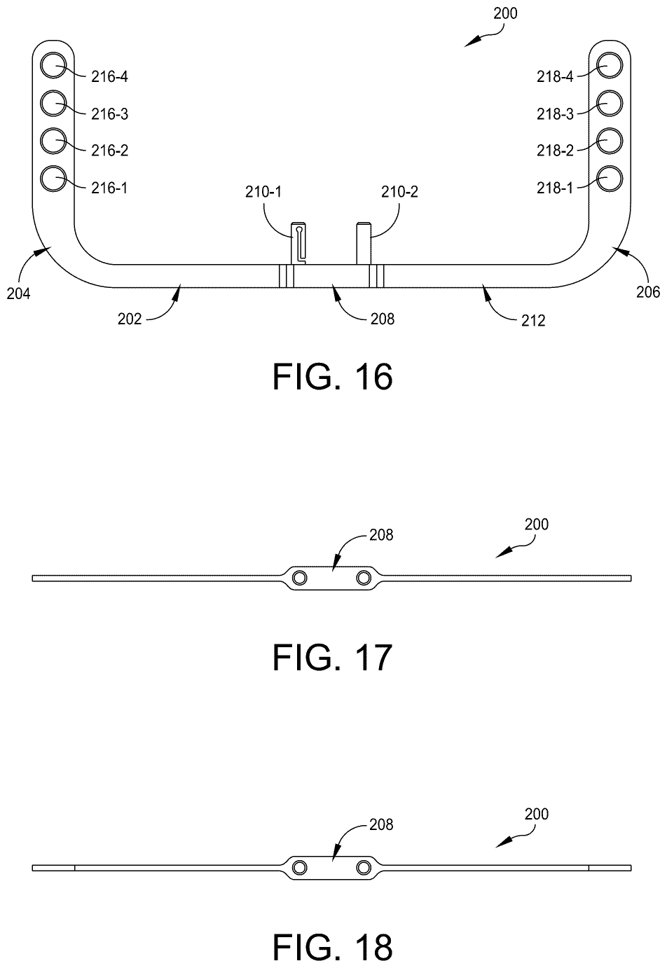

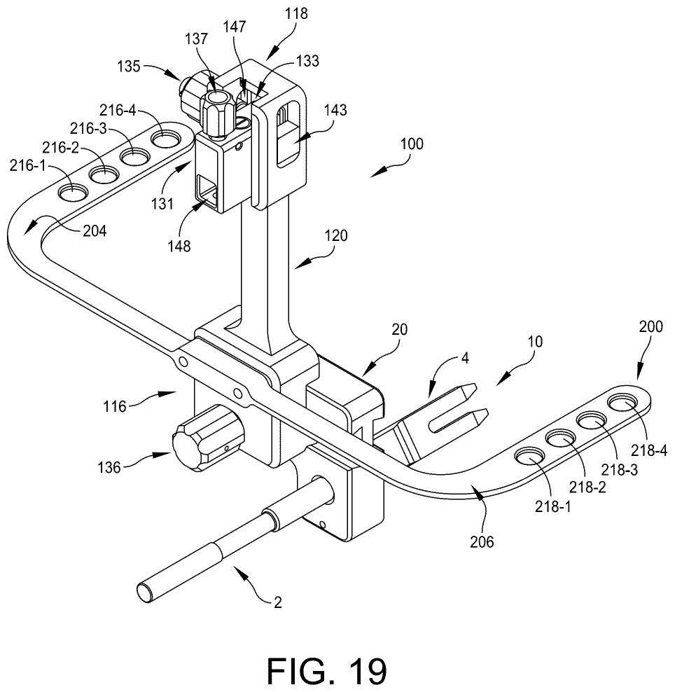

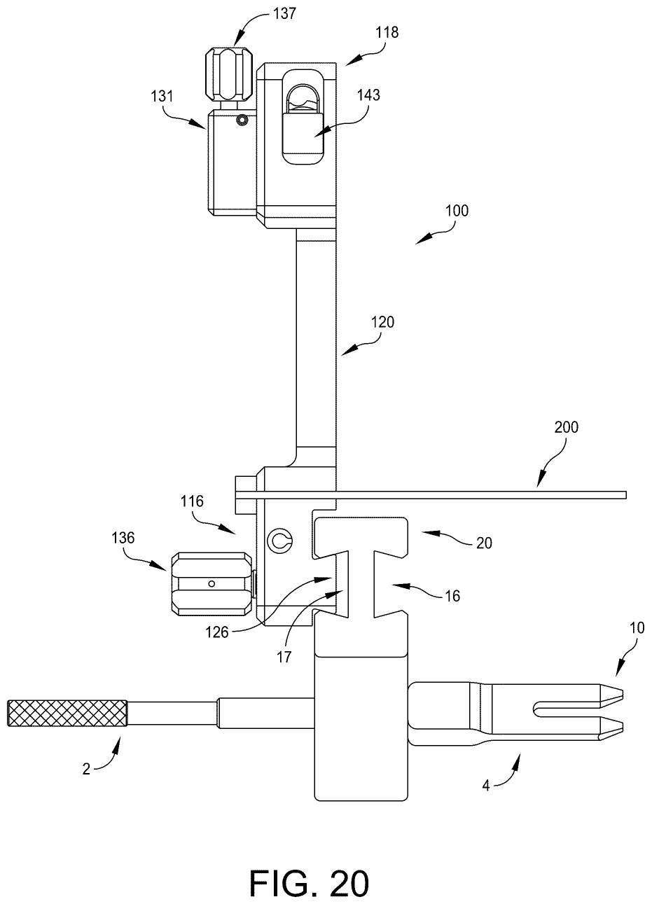

Referring to FIGS. 16-18, angel wing alignment guide 200 may have a generally arcuate or “u-shaped” body with a transverse beam 202 extending between a first arm 204 and a second arm 206. As best seen in FIGS. 17 and 18, the transverse beam 202 may include an enlarged area 208, which may have a width that is greater than a width of a remainder of the transverse beam 202 and/or a width of arms 204, 206. In some embodiments, one or more dowels or protrusions 210-1, 210-2 (collectively, “dowels 210” or “protrusions 210”) extend from a posterior surface 212 of alignment guide 200. Protrusions 210 are sized and configured to be received within at least one hole 138 defined by base 116 of guide 100 for coupling the alignment guide 200 to guide 100 as best seen in FIGS. 19-20. One of ordinary skill in the art will understand that while the protrusions 210 are shown as having a generally circular cross-sectional shape, which a cross section is taken along an axis perpendicular to a longitudinal axis of the protrusions, the protrusions 210 may have other cross-sectional shapes, e.g., triangular, rectangular, etc.

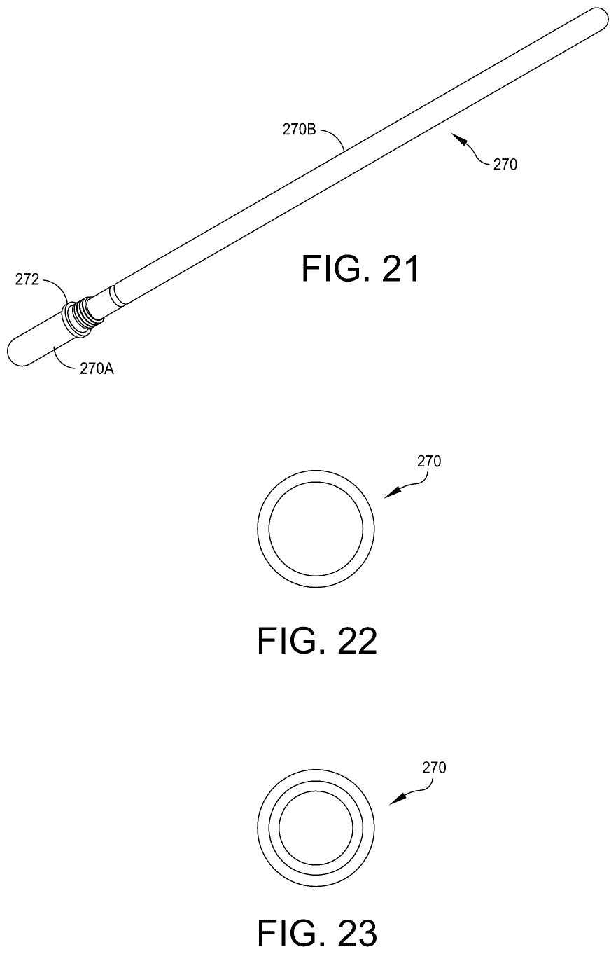

In some embodiments, arms 204 includes one or more holes 216-1, 216-2, 216-3, 216-4 (collectively, “holes 216”), and arm 206 includes one or more holes 218-1, 218-2, 218-3, 218-4 (collectively, “holes 218”). Holes 216, 218 are sized and configured to receive a dowel, pin, rod, or other elongate radiopaque device, such as alignment rod 270 illustrated in FIGS. 21-23. The alignment rod 270 may be coupled to the alignment guide 200 to provide a surgeon with a mediolateral view to approximate an axis of a tibial implant that is to be implanted. The alignment guide 200 can also provide a surgeon with an anteroposterior view to approximate the mechanical axis of a tibial implant. Using alignment guide 200 and alignment rod 270, the surgeon can assess the proper location of a cutting and/or reaming guide with a mechanical and/or anatomic axis of the patient. Although a plurality of holes is shown in FIG. 16 to provide the surgeon with multiple locations at which the alignment rod 270 may be positioned, it should be understood that a single hole may be provided, or one or more slots may be provided along the longitudinal axes of the arms 204, 206 to provide the surgeon with the ability to provide near continuous adjustment of the location of such an alignment rod 270.

In some embodiments, alignment rod 270 has an elongate body that includes a stop collar 272 disposed along its length to divide alignment rod 170 into unequal portions 270A and 270B. As shown in FIGS. 21-23, portion 270A is shorter than portion 270B.

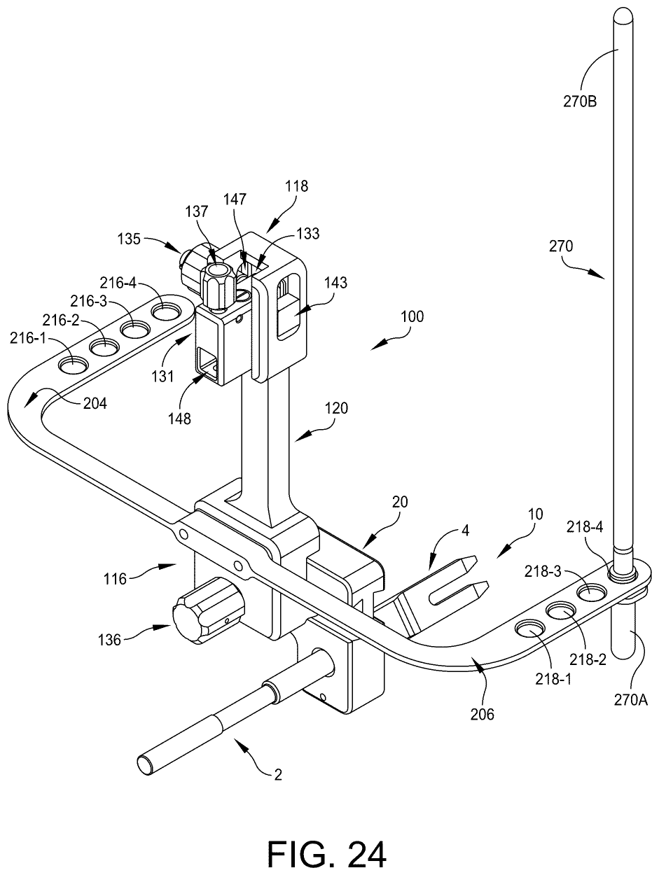

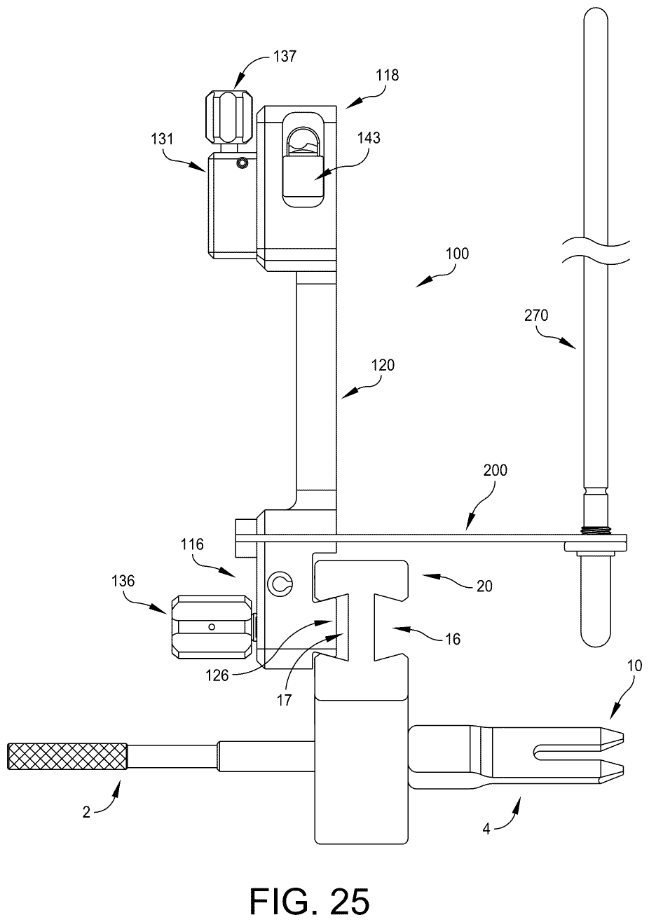

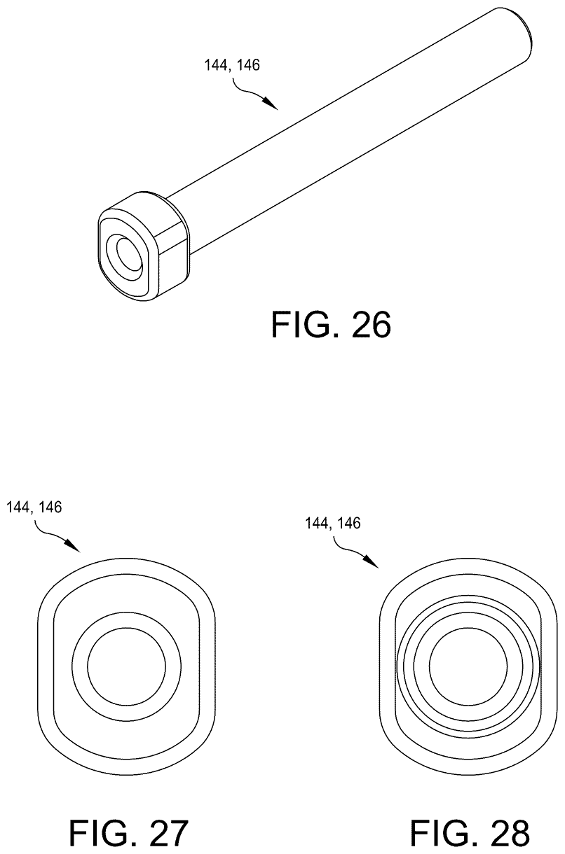

The installation of angel wing alignment guide 200 and alignment rod 270 are described briefly with reference to FIGS. 19-20 and 24-25.

In some embodiments, the angel wing alignment guide 200 is attached to the flexion/extension guide 100 by inserting the angel wing alignment guide protrusions 210 into the at least one hole 138 of base 116. In some embodiments, the position of the angel wing alignment guide 200 is viewed under A/P fluoroscopy to establish coronal alignment, which is typically parallel to the natural joint line.

Portion 270B of alignment rod 270 is inserted through one of the holes 216, 218 of the angel wing alignment guide 200, until stop collar 272 abuts angle wing alignment guide 200. The position of the alignment rod 270 can be viewed under lateral fluoroscopy to establish sagittal rotation, which is typically parallel to a shaft of the tibia 260. Knob 135 may be turned to lock rectangular engagement portion 131 of base 118 in place after the desired alignment is achieved.

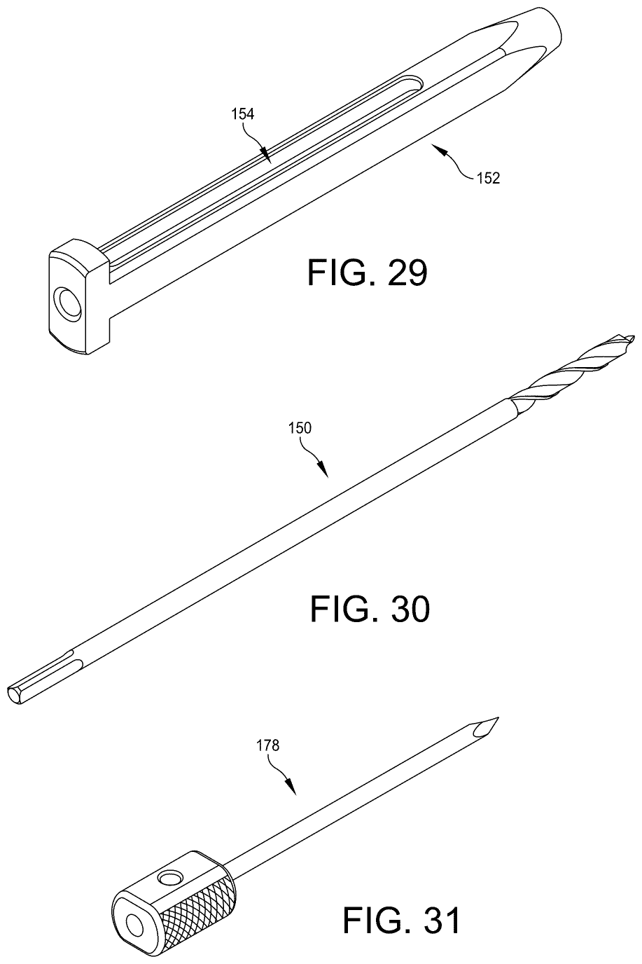

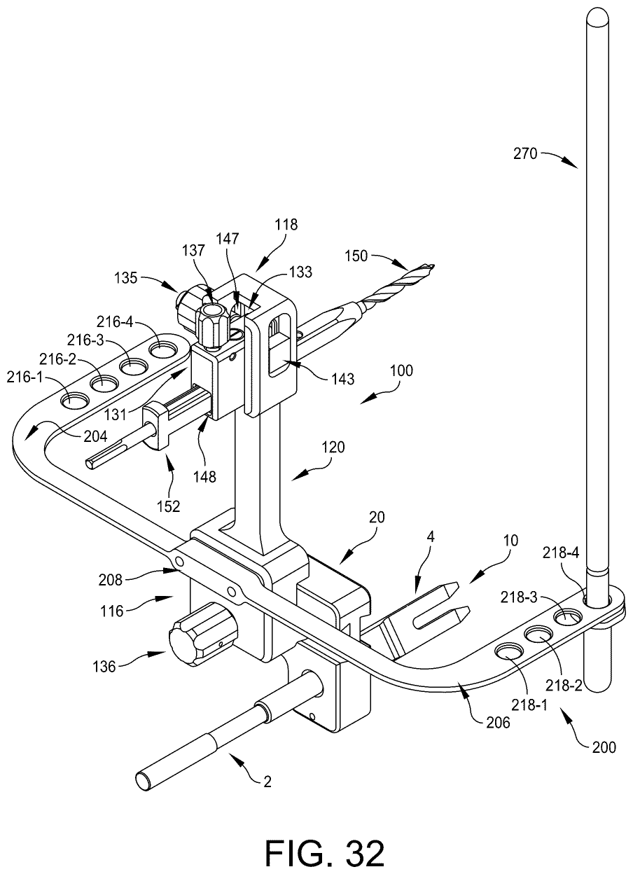

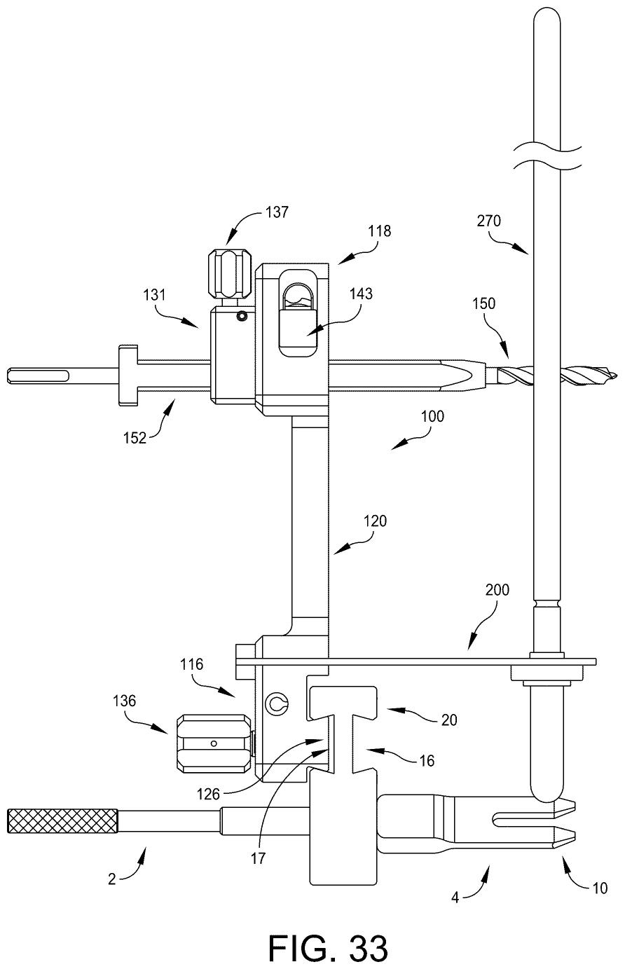

As illustrated in FIGS. 29-30 and 32-33, pin sleeve 152 is inserted into hole 148 of rectangular engagement portion 131 of base 118 of the flexion/extension guide 100. A trocar, such as trocar 178 illustrated in FIG. 31, may be inserted into the pin sleeve 152 to create a “stab wound” or indentation in the bone to prevent skiving for drilling of a percutaneous pin. The trocar 178 is then removed. A guide pin 150 is inserted into pin sleeve 152 and through the tibia 260. Knob 137 may then be turned to allow extension 145 to be inserted into slot 154 of pin sleeve 152 to lock pin 150 in place.

While the flexion/extension guide 100, rotation guide slide 20, angel wing alignment guide 200, alignment rod 270 and pin sleeve 152 are described as being operationally connected to medial gutter fork 10 after it is inserted into the medial gutter of the ankle joint, one of ordinary skill in the art will understand that the flexion/extension guide 100, rotation guide slide 20, angel wing alignment guide 200, alignment rod 270 and pin sleeve 152 may be assembled prior to placing guide hole 18 of rotation guide slide 20 over the distal end 5 of shaft 2 of the medial gutter fork 10. Thus a surgeon may place the entire assembly of flexion/extension guide 100, rotation guide slide 20, angel wing alignment guide 200, alignment rod 270 and pin sleeve 152 over the distal end 5 of shaft 2 of the medial gutter fork 10 through guide hole 18 of rotation guide slide 20. A surgeon may then adjust the alignment of the guides and use knob 136 to lock the mediolateral translation location of rotation guide slide 20 and flexion/extension guide 100. A trocar 178 may then be inserted into the pin sleeve 152 and removed. Guide pin 150 may then be inserted into pin sleeve 152 and through the tibia 260. Knob 135 may then be used to lock rectangular engagement portion 131 in place and knob 137 may be used to allow extension 145 to be inserted into slot 154 of pin sleeve 152 to lock pin 150 in place.

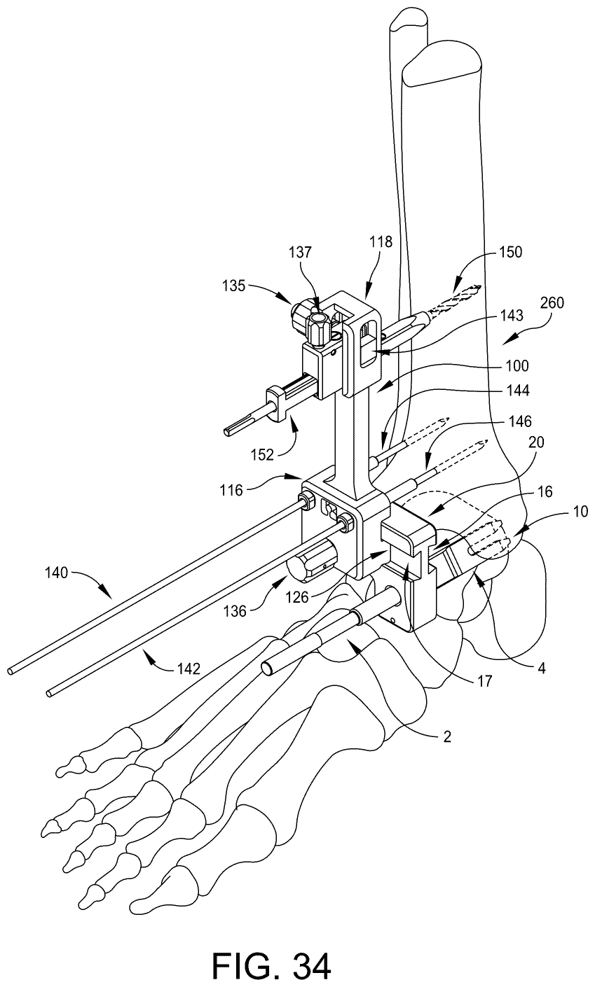

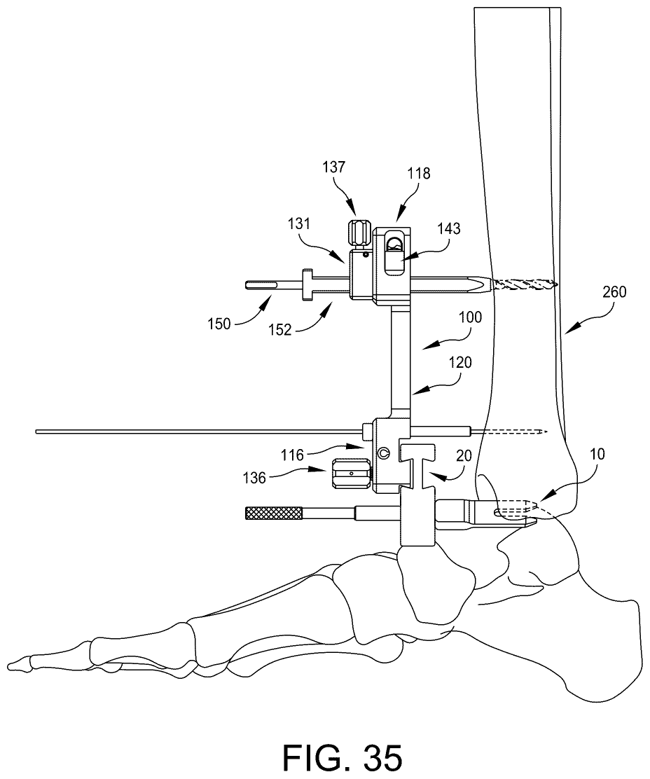

After all adjustments are made, the angel wing alignment guide 200 and alignment rod 270 are removed. As illustrated in FIGS. 34-35, pin sleeves 144, 146 are inserted into at least one hole 138 of base 116 of the flexion/extension guide 100. A trocar, such as trocar 178 illustrated in FIG. 31, may be inserted into each of the pin sleeves 144, 146 to create “stab wounds” or indentation in the bone to prevent skiving for drilling of percutaneous pins. The trocar 178 is then removed.

As illustrated in FIGS. 34-35, pins 140, 142 are inserted into each of the pin sleeves 144, 146 and through both cortices of the tibia 260, which is used for positioning of other structures of the total ankle replacement system. Once the pins 140, 142 are placed, the pin sleeves 144, 146 are removed and knobs 135, 136 and 137 are loosened to aid in the removal of the flexion/extension guide 100. The guide pin 150 and pin sleeve 152 are then removed, followed by the flexion/extension guide 100, rotation guide slide 20 and gutter fork 10, leaving pins 140, 142 in the tibia 260.

The tibia and talus may be further prepared for ankle replacement surgery with an implant using any suitable surgical technique, including the technique disclosed in U.S. Pat. No. 10,136,904, which was incorporated by reference above.

Although the guides, systems, and methods have been described in terms of exemplary embodiments, they are not limited thereto. Rather, the appended claims should be construed broadly, to include other variants and embodiments of the guides, systems, and methods, which may be made by those skilled in the art without departing from the scope and range of equivalents of the guides, systems, and methods.

What is claimed is:

1. A method, comprising:
inserting a first guide above a joint line of a calcaneus and a talus bone;
connecting a second guide to the first guide, the second guide having a first portion extending longitudinally in a first direction and a second portion extending laterally from the first portion in a second direction, wherein the first portion defines a first channel that runs the length of first portion on a first side and defines a second channel on a second side, which is disposed opposite the first side, and the second portion of the second guide defines a guide hole which is configured to engage a shaft of the first guide;
connecting a portion of the second guide to a third guide, the third guide including a body having a first side, a second side, a third side, and a fourth side, the third and fourth sides extending between the first and second sides, wherein the body comprises a first base at said first side, a second base at said second side and an elongated extension between the first and second base, wherein the first base defines at least one first hole and the second base defines at least a second hole, and wherein the body is sized and configured to be installed above a joint line of a calcaneus and a talus bone and approximately mid-way along the longitudinal axis of a tibia;
locking the location of the second guide and third guide about an axis defined by a shaft of the tibia using an adjustment mechanism attached to the third guide;
coupling a fourth guide to the third guide, the fourth guide including first and second arms that are coupled together by a transverse beam and each of the first and second arms of the fourth guide define at least one of a hole or a slot for receiving an elongate radiopaque device therein;
coupling an elongate radiopaque device to the fourth guide through the at least one hole or slot of the fourth guide;
determining the alignment of the fourth guide and elongate radiopaque device using fluoroscopy;
inserting at least one pin sleeve and pin into the second hole of the third guide;
removing the fourth guide and elongate radiopaque device;
inserting pins into the at least one first hole of the third guide and through two cortices of the tibia; and
removing the first, second and third guides.

2. The method of claim 1, wherein coupling the fourth guide to the third guide includes inserting at least one protrusion extending from the transverse beam of the fourth guide into at least one hole defined by the third guide.

3. The method of claim 1, wherein inserting at least one pin into the second hole of the third guide further includes turning an adjustment mechanism located on the second base to lock the pin in place after inserting the pin sleeve and pin into the second hole of the third guide.

4. The method of claim 1, further comprising loosening all adjustment mechanisms attached to the third guide prior to removing the first, second and third guides.

5. A method, comprising:
inserting a first guide above a joint line of a calcaneus and a talus bone;
connecting a portion of a second guide to a third guide, the second guide having a first portion extending longitudinally in a first direction and a second portion extending laterally from the first portion in a second direction, wherein the first portion defines a first channel that runs the length of first portion on a first side and defines a second channel on a second side, which is disposed opposite the first side, and the second portion of the second guide defines a guide hole which is configured to engage a shaft of the first guide;
wherein the third guide includes a body having a first side, a second side, a third side, and a fourth side, the third and fourth sides extending between the first and second sides, wherein the body comprises a first base at said first side, a second base at said second side and an elongated extension between the first and second base, wherein the first base defines at least one first hole and the second base defines at least a second hole, and wherein the body is sized and configured to be installed above a joint line of a calcaneus and a talus bone and approximately mid-way along the longitudinal axis of a tibia;
inserting at least one pin sleeve into the second hole of the third guide;
coupling a fourth guide to the third guide, the fourth guide including first and second arms that are coupled together by a transverse beam and each of the first and second arms of the fourth guide define at least one of a hole or a slot for receiving an elongate radiopaque device therein;
coupling an elongate radiopaque device to the fourth guide through the at least one hole or slot of the fourth guide;
connecting the second guide to the first guide;
locking the location of the second guide and third guide about an axis defined by a shaft of the tibia using an adjustment mechanism attached to the third guide;
determining the alignment of the fourth guide and elongate radiopaque device using fluoroscopy;
inserting at least one pin into the pin sleeve that is inserted into the second hole of the third guide;
removing the fourth guide and elongate radiopaque device;
inserting pins into the at least one first hole of the third guide and through two cortices of the tibia; and
removing the first, second and third guides.

6. The method of claim 5, wherein coupling the fourth guide to the third guide includes inserting at least one protrusion extending from the transverse beam of the fourth guide into at least one hole defined by the third guide.

7. The method of claim 5, wherein inserting at least one pin into the second hole of the third guide further includes turning an adjustment mechanism located on the second base to lock the pin in place after inserting the pin sleeve and pin into the second hole of the third guide.

8. The method of claim 5, further comprising loosening all adjustment mechanisms attached to the third guide prior to removing the first, second and third guides.

9. The method of claim 1, further comprising inserting a trocar through the at least one pin sleeve prior to inserting the at least one pin, the trocar being configured to create a percutaneous indentation in bone to reduce skiving during drilling of the pin.

10. The method of claim 1, wherein connecting the second guide to the first guide further comprises selecting, based on whether the procedure involves a left or a right ankle, one of the first channel or the second channel of the second guide to face away from the tibia before coupling the protrusion of the third guide with the selected channel.

11. The method of claim 1, further comprising, while viewing under anterior-posterior fluoroscopy, confirming that an alignment aperture defined in the first base of the third guide is concentric with a predetermined symmetry marker on the tibia to verify that the fluoroscopic image is a true, non-skewed coronal view before actuating the adjustment mechanism to lock the second guide and the third guide.

12. The method of claim 2, wherein coupling the fourth guide to the third guide further comprises:
a. inserting an alignment rod through one of a plurality of holes in the fourth guide until a stop collar on the alignment rod abuts the fourth guide; and
b. observing the alignment rod under lateral fluoroscopy to confirm that the rod is substantially parallel to a mechanical axis of the tibia so as to establish sagittal rotational alignment.

13. The method of claim 1, further comprising, after inserting the at least one pin sleeve and pin into the second hole of the third guide, rotating an engagement portion that carries the second hole relative to the second base to adjust a flexion or extension angle of the at least one pin with respect to the tibia, and then actuating a second adjustment mechanism to lock the engagement portion in the selected position.

14. The method of claim 5, further comprising, after inserting the pins through two cortices of the tibia, loosening each adjustment mechanism attached to the third guide to facilitate atraumatic removal of the first, second and third guides without disturbing the previously seated pins.

15. A surgical guide system comprising:
(a) a medial gutter fork including a shaft having proximal and distal ends and a head disposed at the distal end, the head configured for insertion into a medial gutter of an ankle joint to provide a rotational reference;
(b) a rotation guide slide having
(i) a first portion extending longitudinally and defining opposed first and second channels, each channel having a tapered cross-section configured to slidingly receive a complementary protrusion, and
(ii) a second portion extending laterally from the first portion and defining a guide hole sized to receive the shaft of the medial gutter fork, the rotation guide slide being mountable on the shaft with either the first channel or the second channel oriented away from the tibia to accommodate right- and left-ankle procedures;
(c) a flexion/extension guide including
(i) a body sized to be positioned above a joint line of a calcaneus and a talus bone and approximately mid-way along a longitudinal axis of a tibia,
(ii) a first base located at a distal end of the body and carrying a longitudinally extending protrusion dimensioned to be slidingly received in the first or second channel of the rotation guide slide,
(iii) a second base located at a proximal end of the body and defining a channel that receives an engagement portion movable between superior and inferior positions, the engagement portion defining a pin hole,
(iv) a first adjustment mechanism operative to lock the protrusion in the selected first or second channel, and
(v) a second adjustment mechanism operative to fix the engagement portion at a selected position within the second base;
(d) a removable pin sleeve sized to be received in the pin hole, the pin sleeve having opposed superior and inferior slots; and
(e) a locking member carried by the engagement portion and selectively engageable with either of the superior or inferior slots to secure a pin inserted through the pin sleeve,
wherein the rotation guide slide, flexion/extension guide, pin sleeve, and locking member cooperate to permit intra-operative adjustment and locking of both (i) mediolateral position about an axis defined by the shaft of the tibia and (ii) flexion or extension angle of a pin disposed in the pin sleeve while the medial gutter fork remains seated in the ankle joint.

16. The system of claim 15, wherein the flexion/extension guide further comprises at least one alignment aperture in the first base configured to provide a visual cue under fluoroscopy for confirming a true coronal view of the tibia.

17. The system of claim 16, wherein the removable pin sleeve is rectangular in cross-section and the pin hole of the engagement portion is correspondingly rectangular to prevent rotational movement of the pin sleeve within the engagement portion.

18. The system of claim 17, wherein the locking member comprises a rotatable extension configured to engage either the superior or inferior slot of the pin sleeve to lock a pin in place in either orientation of the pin sleeve.

19. The system of claim 18, further comprising an alignment guide having a transverse beam and first and second arms, the alignment guide being configured to couple to the first base of the flexion/extension guide via at least one protrusion received in a corresponding hole in the first base.

20. The system of claim 19, wherein at least one of the first and second arms of the alignment guide defines a plurality of holes or slots for receiving an elongate radiopaque device to assist in establishing a mechanical axis of the tibia.

21. The system of claim 20, wherein the elongate radiopaque device comprises a stop collar disposed along its length to limit insertion depth through the alignment guide.

22. The system of claim 21, wherein the medial gutter fork includes a forked head with a pair of prongs configured to engage the medial gutter of the ankle joint for rotational reference.

23. The system of claim 22, wherein the flexion/extension guide, rotation guide slide, and alignment guide are each formed from a radiopaque material to facilitate intra-operative imaging and alignment.
